SEQUENCE LISTING (19 sequences)

SEQ ID NO: 1 - protein, 9 aa, Homo sapiens
TEFTTALQR

SEQ ID NO: 2 - protein, 10 aa, Homo sapiens
LTEQPELANK

SEQ ID NO: 3 - protein, 9 aa, Homo sapiens
VVSQEEIVR

SEQ ID NO: 4 - protein, 13 aa, Homo sapiens
AGAVAEEVLAAIR

SEQ ID NO: 5 - protein, 13 aa, Homo sapiens
TIPIDGNFFTYTR

SEQ ID NO: 6 - protein, 9 aa, Homo sapiens
ILDLIESGK

SEQ ID NO: 7 - protein, 16 aa, Homo sapiens
LLPEYPGVLSDVQEEK

SEQ ID NO: 8 - protein, 25 aa, Homo sapiens
SNSEIICCTTPSLQQLNLQLPLKTK

SEQ ID NO: 9 - protein, 22 aa, Homo sapiens
ETKDGFMFLTDQSYIDVLPEFR

SEQ ID NO: 10 - protein, 24 aa, Homo sapiens
GHFGCVYHGTLLDNDGKKIHCAVK

SEQ ID NO: 11 - protein, 26 aa, Homo sapiens
TKAFFMLDGILSKYFDLIYVHNPVFK

SEQ ID NO: 12 - protein, 21 aa, Homo sapiens
MKAPAVLAPGILVLLFTLVQR

SEQ ID NO: 13 - protein, 20 aa, Homo sapiens
EVFNILQAAYVSKPGAQLAR

SEQ ID NO: 14 - protein, 14 aa, Homo sapiens
GDLTIANLGTSEGR

SEQ ID NO: 15 - protein, 11 aa, Homo sapiens
QIKDLGSELVR

SEQ ID NO: 16 - protein, 26 aa, Homo sapiens
FINFFVGNTINSSYFPDHPLHSISVR

SEQ ID NO: 17 - protein, 18 aa, Homo sapiens
ITDIGEVSQFLTEGIIMK

SEQ ID NO: 18 - protein, 11 aa, Homo sapiens
AFFMLDGILSK

SEQ ID NO: 19 - protein, 9 aa, Homo sapiens
NLNSVSVPR

(19) United States
(12) Patent Application Publication
(10) Pub. No.: US 2018/0172690 A1
(43) Pub. Date: Jun. 21, 2018

(54) METHOD FOR IMPROVED HEPATOCELLULAR CANCER DIAGNOSIS

(71) Applicant: Expression Pathology, Inc., Rockville, MD (US)

(72) Inventors: Shahrooz RABIZADEH, Culver City, CA (US); Todd HEMBROUGH, Gaithersburg, MD (US); Fabiola CECCHI, Washington, DC (US); Christina YAU, San Francisco, CA (US); David KRIZMAN, Gaithersburg, MD (US); Wei-Li Liao, Herndon, VA (US); Sheeno Thyparambil, Frederick, MD (US)

(21) Appl. No.: 15/493,127

(22) Filed: Apr. 20, 2017

Related U.S. Application Data
(60) Provisional application No. 62/325,307, filed on Apr. 20, 2016.
(60) Provisional application No. 62/324,977, filed on Apr. 20, 2016.
(60) Provisional application No. 62/324,964, filed on Apr. 20, 2016.

(51) Int. Cl. G01N 33/574

(52) U.S. Cl. CPC G01N 33/57438; G01N 2560/00; G01N 2333/90203; G01N 2333/90666; G01N 2333/70596; G01N 2333/71

(57) ABSTRACT

Methods are provided for determining the diagnosis of whether a liver mass is a benign hepatocellular adenoma or a pre-malignant hepatocellular dysplastic nodule and/or a malignant hepatocellular carcinoma. Specific protein fragment peptides are precisely detected and quantitated by SRM-mass spectrometry directly in liver mass cells collected from liver mass tissue that was obtained from a patient suffering from the liver mass and compared to reference levels in order to determine if the liver mass is a benign growth or a pre-cancer and/or cancer.

This application claims priority to application Ser. Nos. 62/325,307, 62/324,964 and 62/324,977, all filed Apr. 20, 2016, the contents of each of which are hereby incorporated by reference in their entireties. This application also contains a sequence listing submitted electronically via EFS-web, which serves as both the paper copy and the computer readable form (CRF) and consists of a file entitled “3900_0035C_ST25”, which was created on Jul. 6, 2017 which is 4,324 bytes in size, and which is also incorporated by reference in its entirety.

### INTRODUCTION

New and improved methods for diagnosing hepatocellular (liver) cancer are provided. The methods provide an assay for measuring levels of specific proteins in tissue from patients suspected of having hepatocellular cancer. More specifically, quantitative assays for the proteins MET, DHFR, MDR1, and ALDHA1 are used to determine if a liver tissue mass removed from a patient is a benign hepatocellular adenoma or a pre-malignant hepatocellular dysplastic nodule/hepatocellular malignant tumor. Current methods to distinguish these liver masses relies on subjective histological analysis by a trained clinical histologist/pathologist and it is found that there is significant variation in the results of this method because no biochemical measure exists to make objective distinctions between the benign and pre-malignant/malignant states. The methods described herein solve the problem of subjectivity in the results by providing an objective, quantitative, statistically significant biochemical assay to determine what type of liver mass is present in the liver of a patient. When a patient presents with a liver mass it is very important to determine the type of the mass because the decision on how best to treat such a patient differs depending on the nature of the cellular growth present in the mass.

If a liver mass is determined to be a benign hepatocellular adenoma the curative treatment is complete surgical resection where all symptomatic benign hepatocellular adenomas should be resected, regardless of size. No follow-up chemotherapy treatment is required and, once resected, the patient is considered cured of benign hepatocellular adenomas. However, asymptomatic benign hepatocellular adenomas smaller than 5 centimeters are not surgically resected and generally managed with close monitoring using MRI as the preferred choice of imaging. Yearly ultrasound imaging and an assessment of serum AFP levels is a consideration in all patients with benign hepatocellular adenomas, especially those with multiple lesions or single lesions greater than 5 cm in diameter who do not undergo surgical resection. In this case such patients will not be harmed by these lesions and generally speaking live a normal life.

However, if a liver mass is determined to be a pre-malignant hepatocellular dysplastic nodule or a fully malignant hepatocellular carcinoma, the initial treatment involves complete surgical resection of each lesion regardless of size followed by either chemotherapy for the patient or close monitoring in order to determine if and/or when further treatment becomes necessary. Pre-malignant hepatocellular dysplastic nodules eventually progress to fully malignant hepatocellular carcinoma and thus there is a need to treat the pre-malignant and malignant hepatocellular lesions much more aggressively than a benign hepatocellular adenoma. Thus there is a great need to determine if a liver mass is a benign hepatocellular adenoma or a much more dangerous dysplastic nodule or malignant carcinoma.

Quantitative expression of MET, DHFR, MDR1, and ALDHA1 proteins in normal liver tissue and in an abnormal liver mass is determined by quantitating a specified fragment peptide from each protein using mass spectrometry. The specified MET, DHFR, MDR1, and ALDHA1 fragment peptides are detected using mass spectrometry-based Selected Reaction Monitoring (SRM), also referred to as Multiple Reaction Monitoring (MRM), and referred to herein as an SRM/MRM assay. An SRM/MRM assay is used to detect the presence and quantitatively measure the amount of the specified fragment peptides, directly in cells procured from cancer patient tissue, such as, for example formalin fixed cancer tissue. The amount of the specific peptides is then used to quantitate the amount of intact MET, DHFR, MDR1, and ALDHA1 proteins in the tumor sample. Precise liver mass diagnosis and treatment strategies are then determined and implemented to treat a patient's hepatocellular disease based on specified levels of the MET, DHFR, MDR1, and ALDHA1 proteins expressed in hepatocellular cells from a liver mass.

### SUMMARY OF THE INVENTION

Methods are provided for diagnosing a liver mass comprising:

(a) detecting expression of a specified MET fragment peptide, a specified DHFR fragment peptide, a specific MDR1 fragment peptide, and a specified ALDHA1 fragment peptide, and quantifying the level of a specified MET fragment peptide, a specified DHFR fragment peptide, a specific MDR1 fragment peptide, and a specified ALDHA1 fragment peptide in a protein digest prepared from a liver mass obtained from a patient suffering from an abnormal liver mass, or masses, and calculating the level of the MET fragment peptide, the DHFR fragment peptide, the MDR1 fragment peptide, and the ALDHA1 fragment peptide in cells obtained from one or more abnormal liver masses by selected reaction monitoring using mass spectrometry;

(b) comparing the level of the MET fragment peptide to a reference level, and

(c) comparing the level of the DHFR fragment peptide to a reference level, and

(d) comparing the level of the MDR1 fragment peptide to a reference level, and

(e) comparing the level of the ALDHA2 fragment peptide to a reference level, and

(f) determining if an abnormal liver mass is a benign hepatocellular adenoma, a pre-malignant hepatocellular dysplastic nodule, or a malignant hepatocellular carcinoma.

In these methods the reference level of the MET fragment peptide may be, for example, 291 amol/μg, +/−290 amol/μg, +/−150 amol/μg, +/−100 amol/μg, +/−50 amol/μg, or +/−25 amol/μg, of biological sample protein analyzed. The reference level of the MET fragment peptide may also be, for example, 187 amol/μg, +/−186 amol/μg, +/−150 amol/μg, +/−100 amol/μg, +/−50 amol/μg, or +/−25 amol/μg.

In these methods the reference level of the DHFR fragment peptide may be, for example, 301 amol/μg, +/−300 amol/μg, +/−150 amol/μg, +/−100 amol/μg, +/−50 amol/μg, or +/−25 amol/μg, of biological sample protein analyzed. The reference level of the DHFR fragment peptide may also be, for example, 162 amol/μg, +/−161 amol/μg, +/−150 amol/μg, +/−100 amol/μg, +/−50 amol/μg, or +/−25 amol/μg.

In these methods the reference level of the MDR1 fragment peptide may be, for example, 81 amol/μg, +/−88 amol/μg, +/−50 amol/μg, or +/−25 amol/μg, of biological sample protein analyzed. The reference level of the MDR1 fragment peptide may also be, for example, 304 amol/μg, +/−303 amol/μg, +/−150 amol/μg, +/−100 amol/μg, +/−50 amol/μg, or +/−25 amol/μg.

In these methods the reference level of the ALDHA1 fragment peptide may be, for example, 20,586 amol/μg, +/−20585 amol/μg, +/−100 amol/μg, +/−50 amol/μg, or +/−25 amol/μg, of biological sample protein analyzed. The reference level of the MDR1 fragment peptide may also be, for example, 32,500 amol/μg, +/−32,499 amol/μg+/−200 amol/μg, +/−150 amol/μg, +/−100 amol/μg, +/−50 amol/μg, or +/−25 amol/μg.

Also provided are methods for measuring the level of the human multidrug resistance protein 1 (MDR1) protein in a human biological sample of formalin-fixed tissue, including detecting and/or quantifying the amount of at least one MDR1 fragment peptide in a protein digest prepared from the human biological sample using mass spectrometry; and calculating the level of MDR1 protein in the sample; where the MDR1 fragment peptide is the peptide of SEQ ID NO:3 or SEQ ID NO:4, and where the level is a relative level or an absolute level.

Further provided are methods for measuring the level of the human Aldehyde Dehydrogenase 1 isoform A1 (ALDHA1) protein in a human biological sample of formalin-fixed tissue, including detecting and/or quantifying the amount of at least one ALDHA1 fragment peptide in a protein digest prepared from the human biological sample using mass spectrometry; and calculating the level of ALDHA1 protein in the sample; where the ALDHA1 fragment peptide is the peptide of SEQ ID NO:5 or SEQ ID NO:6, and where the level is a relative level or an absolute level.

The mass spectrometry may be, for example, tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry. The mode of mass spectrometry used may be, for example, Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

As shown in Table 1, in these methods the specified MET peptide may have the amino acid sequence as set forth as SEQ ID NO: 1. The specified DHFR peptide may have the amino acid sequence as set forth as SEQ ID NO:2. The specified MDR1 peptide may have the amino acid sequence as set forth as SEQ ID NO:3 or SEQ ID NO:4. The specified ALDHA1 peptide may have the amino acid sequence as set forth as SEQ ID NO:5 or SEQ ID NO:6. The analyzed biological sample may be, for example a cell, collection of cells, or a solid tissue derived from a liver mass obtained from a patient suffering from liver disease. The liver mass removed from the patient and to be analyzed may be formalin fixed solid tissue, and may be paraffin embedded tissue.

In these methods quantifying the specified MET, DHFR, MDR1, and ALDHA1 fragment peptides may include determining the amount of the MET, DHFR, MDR1, and ALDHA1 peptides in the sample by comparing to a spiked internal standard peptide of known amount, where both the native peptide in the biological sample and the internal standard peptide corresponds to the same amino acid sequence of the MET, DHFR, MDR1, and ALDHA1 fragment peptides as shown in SEQ ID NO:1, SEQ ID NO:2, SEQ ID NO:3, SEQ ID NO:4, SEQ ID NO:5, and SEQ ID NO:6. The internal standard peptide may be, for example, an isotopically labeled peptide. The isotopically labeled internal standard peptide may comprise one or more heavy stable isotopes selected from ¹⁸O, ¹⁷O, ¹⁵N, ¹³C, ²H or combinations thereof.

Detecting and quantitating the specified MET, DHFR, MDR1, and ALDHA1 fragment peptides can be combined with detecting and quantitating other peptides from other proteins in a multiplex format so that the diagnostic decision about the liver mass is based upon specific levels of the specified MET, DHFR, MDR1, and ALDHA1 fragment peptides in combination with other peptides/proteins in the biological sample.

In the methods for detecting MET, DHFR, MDR1, and ALDHA fragment peptides the method may further include the step of fractionating the protein digest prior to detecting and/or quantifying the amount of the fragment peptides. Quantifying the fragment peptides may include comparing the amount of the fragment peptides in one biological sample to the amount of the same fragment peptides in a different and separate biological sample, or may include determining the amount of the fragment peptides in a biological sample by comparison to an added internal standard peptide of known amount, where the fragment peptides in the biological sample is compared to an internal standard peptides having the same amino acid sequence; and where the internal standard peptides are isotopically labeled peptides.

In these methods detecting and/or quantifying the amount of the fragment peptides in the protein digest indicates the presence of the corresponding modified or unmodified protein and an association with a specific liver mass in the patient, and the results may be correlated to the diagnosis of the liver mass. The correlating step may be combined with detecting and/or quantifying the amount of other proteins or peptides from other proteins in a multiplex format to provide additional information about the diagnosis of the liver mass.

After the measurement and, optionally, the correlating step, the patient from whom the biological sample was obtained is treated according to the treatment decisions put forth for a specific diagnostic stage of the liver mass as determined by the amount of the MET, DHFR, MDR1, and ALDHA fragment peptides or the level of the proteins, as described herein.

### BRIEF DESCRIPTION OF THE DRAWINGS

FIG. 1 shows the unsupervised clustering results of quantitative proteomic analysis by mass spectrometry-selected reaction monitoring of 24 proteins. 22 liver mass formalin fixed paraffin embedded samples (9 Malignant, 5 Pre-malignant, and 8 benign) were analyzed by SRM methodology for 24 different proteins. Tissue samples were clustered based on the 24 protein biomarker profiles using the Ward minimum variance method with squared Euclidean distance. Clustering (average linkage) of proteins based on Pearson correlation across samples was also performed. Data was median-centered and scaled to standard deviation of 1 and displayed in a heatmap. Differences in sample composition between clusters are assessed using the Fisher's Exact test. When samples are clustered based on their quantitative protein profiles, two main clusters are observed (FIG. 1A). The two clusters have significant differences in their sample composition (FIG. 1B), where the majority of premalignant and malignant samples co-clusters in Cluster 1 and all but one of the benign samples fall into Cluster 2

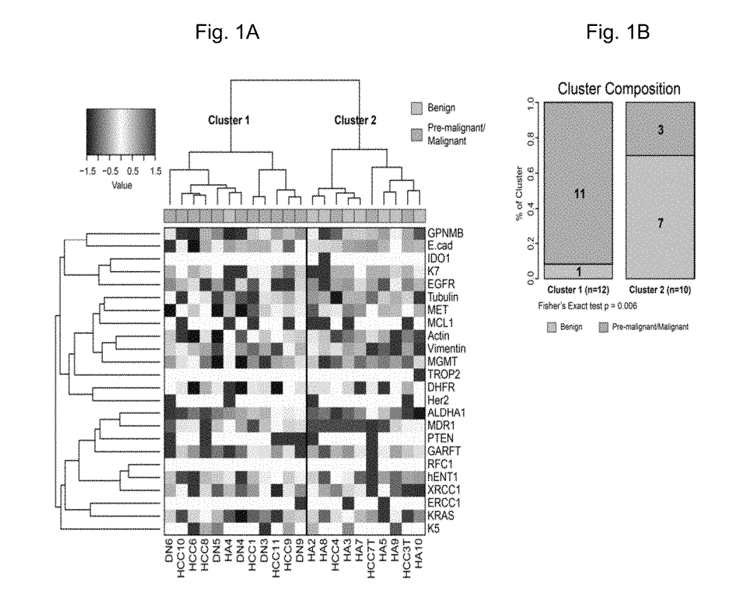

FIG. 2 shows the results in quantitation of 4 proteins that are significantly differentially expressed between benign hepatocellular adenomas and the grouped pre-malignant hepatocellular dysplastic nodules and malignant hepatocellular carcinomas. Since unsupervised clustering analysis suggest that pre-malignant samples shares a similar protein expression profile as the malignant samples, these two groups were combined and a t-test was used to identify proteins with significant differences between this combined group and the benign cases. Significance threshold is set at p<0.05 without multiple hypothesis testing correction. Table 2 shows the group means among the benign samples and the combined pre-malignant/malignant group and the t-test p values. 4 proteins (MET, DHFR, MDR1, and ALDHA1) show statistically significant differences in protein levels between these dichotomized groups, where MDR1 and ALDHA1 levels appears higher in the pre-malignant/malignant group while DFHR and MET levels appears lower.

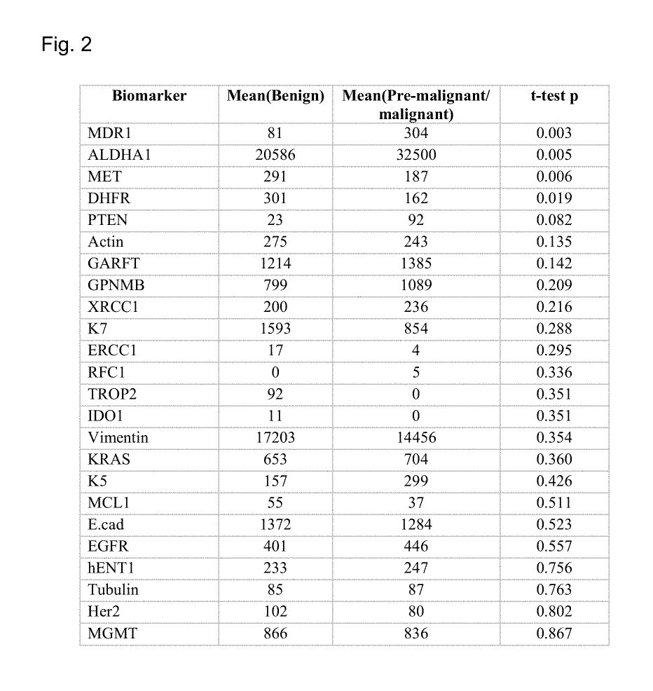

FIG. 3 graphically shows the mean quantitative values for each of the 4 statistically significant proteins as shown in Table 2 and demonstrates the statistical significance compared to the clustering results. The mean values for MDR1 (FIG. 3A) and ALDHA1 (FIG. 3B) levels appears higher in the pre-malignant/malignant group while the mean values for DFHR (FIG. 3D) and MET (FIG. 3C) levels appears lower.

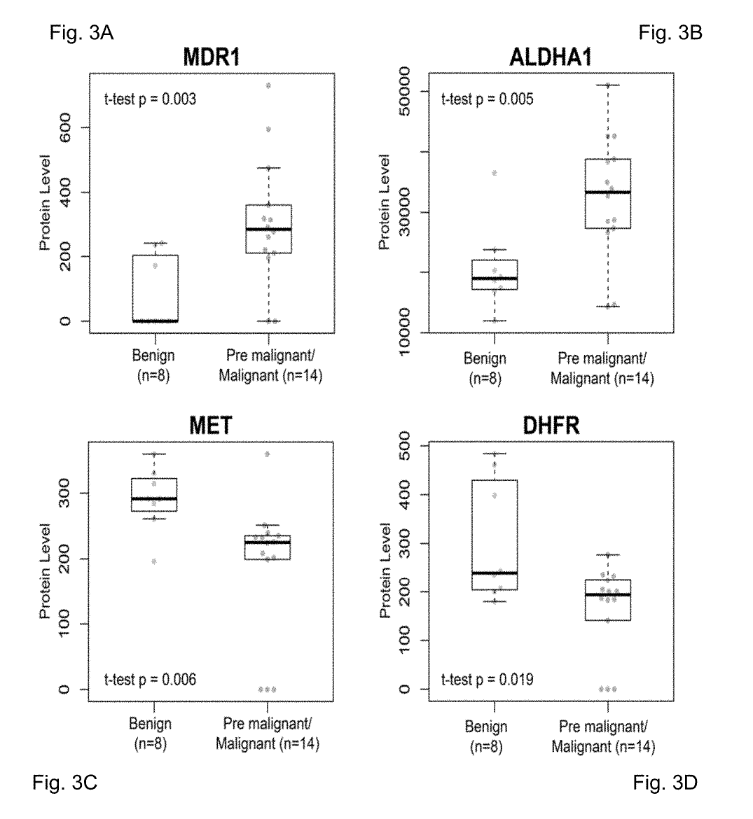

FIG. 4 shows results of a Diagonal Linear Discriminant Analysis (DLDA) using the protein levels of MET, MDR1, DHFR and ALDHA1 to see how well these 4 proteins predict benign vs. premalignant/malignant cases. The average 3-fold cross-validation error is computed as an estimate of the robustness of the discrimination. In addition, to further assess discrimination accuracy, the protein data were permuted and computed with classification accuracy over 1000 iterations; and a permutation based p value for the DLDA accuracy is computed as the number of iterations where the classifier has higher (or equal) accuracy in the randomly permuted data than the actual data+1 divided by the number of iterations+1.

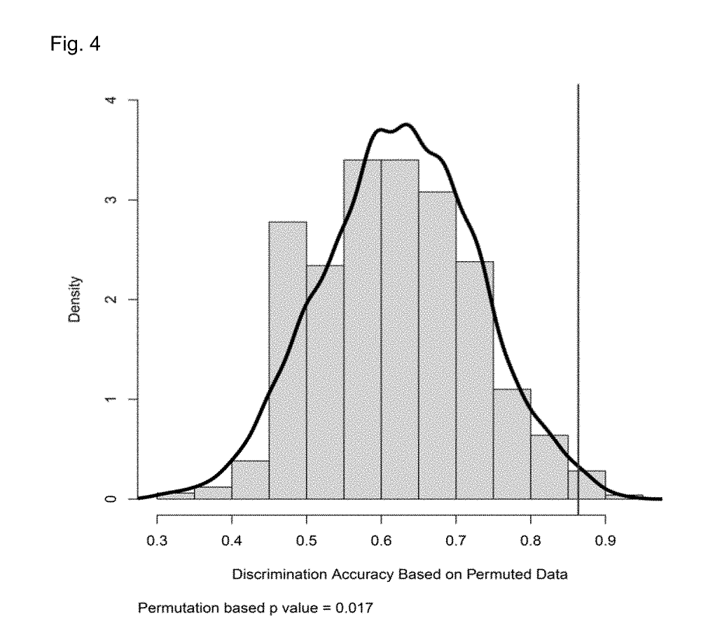

The results show that DLDA correctly classifies all 8 benign cases as benign and 11 of 14 the 14 pre-malignant/malignant cases as pre-malignant/malignant. 3 malignant cases (HCC1, HCC3T and HCC4) were misclassified; and of these 3 misclassified malignant cases, 2 co-clustered with benign samples in the unsupervised clustering analysis. The average 3-fold cross-validation error of a diagonal linear discriminant analysis based on the four biomarkers is ˜17%. Results show the distribution of DLDA classification accuracy across the randomly permuted sample, with the vertical line indicating DLDA accuracy based on real data. The permutation based p value of DLDA is 0.017, suggesting that classification using the actual data is significantly better than one would expect based on random data.

### DETAILED DESCRIPTION

Methods are provided for providing an accurate classification and diagnosis for a liver mass remove from a patient suffering from liver disease. Specifically, diagnostic methods are provided for measuring expression of the combination of the MET, ALDHA1, DHFR, and MDR1 proteins in a protein lysate sample prepared from cells obtained from a liver mass. The liver mass (benign or pre-malignant or malignant) sample is advantageously a formalin-fixed sample. Using an SRM/MRM assay that measures specific peptide fragments, and particular characteristics about the peptides, the amount of the MET, ALDHA1, DHFR, and MDR1 proteins in cells derived from formalin fixed paraffin embedded (FFPE) tissue is determined.

The presence and/or quantitative levels of MET and DHFR are measured using specific fragment peptides derived from the proteins as described in U.S. application Ser. No. 13/976,956 (filed Dec. 27, 2010), U.S. application 62/266,441, (filed Dec. 11, 2015), and U.S. application Ser. No. 15/376,527, (filed Dec. 12, 2016), the contents of each of which are hereby incorporated by reference in their entireties.

SRM/MRM Assays for MET, ALDHA1, DHFR, and MDR1

The peptide fragments derive from the full-length proteins; the specific peptide sequences that are used for detecting and quantitating the MET, ALDHA1, DHFR, and MDR1 proteins are shown in Table 1:

| SEQ ID | Protein | Peptide Sequence |
|---|---|---|
| SEQ ID NO: 1 | MET | TEFTTALQR |
| SEQ ID NO: 2 | DHFR | LTEQPELANK |
| SEQ ID NO: 3 | MDR1 | VVSQEEIVR |
| SEQ ID NO: 4 | MDR1 | AGAVAEEVLAAIR |
| SEQ ID NO: 5 | ALDHA1 | TIPIDGNFFTYTR |
| SEQ ID NO: 6 | ALDHA1 | ILDLIESGK |

Advantageously, the peptide of SEQ ID NO:3 is used for detecting MDR1 and the peptide of SEQ ID NO:6 is used for detecting ALDHA1.

Detection and accurate quantitation of specific peptides from any of the proteins in digests of FFPE tissue is highly unpredictable, due to the random protein crosslinking that occurs during formalin fixation of proteins. Surprisingly, however, it has been found that these specific MET, DHFR, MDR1, and ALDHA1 fragment peptides can be reliably detected and quantitated simultaneously in digests prepared from FFPE samples of tumor tissue. See, for example, U.S. Pat. No. 9,139,864, the contents of which are hereby incorporated by reference in their entirety.

The SRM/MRM assays can be used to measure relative or absolute quantitative levels of specific peptides from MET, DHFR, MDR1, and ALDHA1 and therefore provide a means of measuring by mass spectrometry the amount of each protein in a given protein preparation obtained from a biological sample. The proteins can be measured individually or in any given combination. Suitable combinations for measurement include:

MET, DHFR, MDR1, and ALDHA1

DHFR, MDR1, and ALDHA1

MET, MDR1, and ALDHA1

MET, DHFR, and ALDHA1

MET, DHFR, and MDR1,

MET and ALDHA1

MET and DHFR

MET and MDR1

DHFR and ALDHA1

DHFR and MDR1 and

MDR1, and ALDHA1

The SRM/MRM assay(s) can measure these peptides directly in complex protein lysate samples prepared from cells procured from patient tissue samples, such as formalin fixed cancer patient tissue. Methods of preparing protein samples from formalin-fixed tissue are described in U.S. Pat. No. 7,473,532, the contents of which are hereby incorporated by reference in their entirety. The methods described in U.S. Pat. No. 7,473,532 may conveniently be carried out using Liquid Tissue reagents and protocol available from Expression Pathology Inc. (Rockville, Md.).

The most widely and advantageously available form of tissue, and liver mass tissue, from patients is formalin fixed, paraffin embedded tissue. Formaldehyde/formalin fixation of surgically removed tissue is by far the most common method of preserving cancer tissue samples worldwide and is the accepted convention in standard pathology practice. Aqueous solutions of formaldehyde are referred to as formalin. “100%” formalin consists of a saturated solution of formaldehyde (about 40% by volume or 37% by mass) in water. A small amount of stabilizer, usually methanol, is added to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature.

The assay described herein measures relative or absolute levels of the specific unmodified peptides from MET, DHFR, MDR1, and/or ALDHA1. Relative quantitative levels of each protein are determined by the SRM/MRM methodology for example by comparing SRM/MRM signature peak areas (e.g., signature peak area or integrated fragment ion intensity) of either one, or both, of the two fragment peptides derived from ALDHA1 in different samples. Alternatively, it is possible to compare multiple SRM/MRM signature peak areas for one or both of the signature peptides, where each peptide has its own specific SRM/MRM signature peak, to determine the relative content of each measured protein in one biological sample with the content of those same proteins in one or more additional or different biological samples. In this way, the amount of a particular peptide, or peptides, from the measured protein(s), and therefore the amount of the protein(s), is determined relative to the same peptide, or peptides, across 2 or more biological samples under the same experimental conditions. In addition, relative quantitation can be determined for a given peptide, or peptides, from a given protein within a single sample by comparing the signature peak area for that peptide by SRM/MRM methodology to the signature peak area for another and different peptide, or peptides, from a different protein, or proteins, within the same protein preparation from the biological sample. In this way, the amount of a particular peptide from a designated protein, and therefore the amount of that protein, is determined relative one to another within the same sample.

These approaches generate quantitation of an individual peptide, or peptides, from a measured protein to the amount of another peptide, or peptides, between samples and within samples wherein the amounts as determined by signature peak area are relative one to another, regardless of the absolute weight to volume or weight to weight amounts of the selected peptide in the protein preparation from the biological sample. Relative quantitative data about individual signature peak areas between different samples are normalized to the amount of protein analyzed per sample. Relative quantitation can be performed across many peptides from multiple proteins and one or more of the designated proteins simultaneously in a single sample and/or across many samples to gain insight into relative protein amounts, such as one peptide/protein with respect to other peptides/proteins.

Absolute quantitative levels of a measured protein are determined by, for example, the SRM/MRM methodology whereby the SRM/MRM signature peak area of at least one peptide from a measured protein in one biological sample is compared to the SRM/MRM signature peak area of a corresponding spiked internal standard or standards. Advantageously, the internal standard is a synthetic version of the same exact peptide derived from the measured protein that contains one or more amino acid residues labeled with one or more heavy isotopes. Such isotope labeled internal standards are synthesized so that when analyzed by mass spectrometry a standard generates a predictable and consistent SRM/MRM signature peak that is different and distinct from the native peptide signature peak and which can be used as a comparator peak. Thus when the internal standard is spiked into a protein preparation from a biological sample in known amounts and analyzed by mass spectrometry, the SRM/MRM signature peak area of the native peptide is compared to the SRM/MRM signature peak area of the internal standard peptide, and this numerical comparison indicates either the absolute molarity and/or absolute weight of the native peptide present in the original protein preparation from the biological sample. Absolute quantitative data for fragment peptides are displayed according to the amount of protein analyzed per sample. Absolute quantitation can be performed across many peptides, and thus proteins, simultaneously in a single sample and/or across many samples to gain insight into absolute protein amounts in individual biological samples and in entire cohorts of individual samples.

The SRM/MRM assay method can be used to aid diagnosis of the stage of cancer, for example, directly in patient-derived tissue, such as formalin fixed tissue, and to aid in determining which therapeutic agent would be most advantageous for use in treating that patient. Cancer tissue that is removed from a patient either through surgery, such as for therapeutic removal of partial or entire tumors, or through biopsy procedures conducted to determine the presence or absence of suspected disease, is analyzed to determine whether or not a specific protein, or proteins, and which forms of proteins, are present in that patient tissue. Moreover, the expression level of a protein, or multiple proteins, can be determined and compared to a “normal” or reference level found in healthy tissue. Normal or reference levels of a measured protein found in healthy tissue may be derived from, for example, the relevant tissues of one or more individuals that do not have cancer. Alternatively, normal or reference levels may be obtained for individuals with cancer by analysis of relevant tissues not affected by the cancer. Assays of protein levels from a measured protein can also be used to diagnose the stage of cancer in a patient or subject diagnosed with cancer by employing the protein levels. The level of an individual peptide derived from a measured protein such as, for example, ALDHA1 is defined as the molar amount of the peptide determined by the SRM/MRM assay per total amount of protein lysate analyzed. Information regarding a designated protein such as ALDHA1 can thus be used to aid in determining the stage or grade of a cancer by correlating the level of the protein(s) (or fragment peptides from the protein) with levels observed in normal tissues.

Once the quantitative amount of a measured protein has been determined in the cancer cells, that information can be matched to a list of therapeutic agents (chemical and biological) developed to specifically treat cancer tissue that is characterized by, for example, abnormal expression of that measured protein. Matching information from a protein assay to a list of therapeutic agents that specifically targets, for example, the designated protein or cells/tissue expressing the protein, defines what has been termed a personalized medicine approach to treating disease. The assay methods described herein form the foundation of a personalized medicine approach by using analysis of proteins from the patient's own tissue as a source for diagnostic and treatment decisions.

In principle, any predicted peptide derived from a designated protein prepared, for example, by digesting with a protease of known specificity (e.g. trypsin), can be used as a surrogate reporter to determine the abundance of a designated protein in a sample using a mass spectrometry-based SRM/MRM assay. Similarly, any predicted peptide sequence containing an amino acid residue at a site that is known to be potentially modified in the designated protein also might potentially be used to assay the extent of modification of the designated protein in a sample.

Suitable fragment peptides derived from a designated protein may be generated by a variety of means including by the use of the Liquid Tissue protocol provided in U.S. Pat. No. 7,473,532. The Liquid Tissue protocol and reagents are capable of producing peptide samples suitable for mass spectroscopic analysis from formalin fixed paraffin embedded tissue by proteolytic digestion of the proteins in the tissue/biological sample. In the Liquid Tissue protocol the tissue/biological is heated in a buffer for an extended period of time (e.g., from about 80° C. to about 100° C. for a period of time from about 10 minutes to about 4 hours) to reverse or release protein cross-linking. The buffer employed is a neutral buffer, (e.g., a Tris-based buffer, or a buffer containing a detergent). Following heat treatment the tissue/biological sample is treated with one or more proteases including, but not limited to, trypsin, chymotrypsin, pepsin, and endoproteinase Lys-C, for a time sufficient to disrupt the tissue and cellular structure of said biological sample. The result of the heating and proteolysis is a liquid, soluble, dilutable biomolecule lysate.

Surprisingly, it has been found that many potential peptide sequences from the measured proteins are unsuitable or ineffective for use in mass spectrometry-based SRM/MRM assays for reasons that are not immediately evident. As it was not possible to predict the most suitable peptides for MRM/SRM assay, it was necessary to experimentally identify peptides in actual Liquid Tissue lysates to develop a reliable and accurate SRM/MRM assay for each measured protein. While not wishing to be bound by any theory, it is believed that some peptides might, for example, be difficult to detect by mass spectrometry because they do not ionize well or produce fragments distinct from other proteins. Peptides may also fail to resolve well in separation (e.g., liquid chromatography), or may adhere to glass or plastic ware.

Improved Methods of Treating Liver Cancer

Results from the SRM/MRM assay can be used to correlate accurate and precise quantitative levels of the MET, DHFR, MDR1, and ALDHA1 proteins within the specific liver mass or liver cancer of the patient from whom the tissue was collected and preserved. This not only provides diagnostic information about the cancer, but also permits a physician or other medical professional to determine appropriate therapy for the patient. In this case, utilizing this assay can provide information about specific levels of MET, DHFR, MDR1, and ALDHA1 protein expression in liver mass tissue from a patient and makes it possible to determine if the liver mass is a benign hepatocellular adenoma which can be cured with surgical resection or if the liver mass is a pre-malignant hepatocellular dysplastic nodule or malignant hepatocellular carcinoma whereby the patient requires a more aggressive approach to treatment which likely includes some form of chemotherapy.

Because there are 2 very different strategies to treating these 2 different groups of liver disease patients, this described assay performs an essential function in order to inform a clinician of the patient diagnosis which aids in patient treatment decisions and patient management.

MET, also known as the Hepatocyte Growth Factor Receptor, is a growth factor receptor that is involved in the division and growth of hepatocytes. It functions by binding hepatocyte growth factor on the hepatocyte cell surface and sends a signal into the cell nucleus to divide. The MET protein is overexpressed in many cancers.

DHFR (dihydrofolate reductase) is a 187 amino acid enzyme that reduces dihydrofolic acid to tetrahydrofolic acid, using NADPH as electron donor. DHFR has a critical role in regulating the amount of tetrahydrofolate in the cell which, along with its derivatives, is essential for purine and thymidylate synthesis and which are important for cell proliferation and growth.

MDR1 (multidrug resistance protein 1), also known as permeability glycoprotein, p-glycoprotein 1 or CD243, is an important protein of the cell membrane that pumps many foreign substances out of cells, including cancer drugs. More formally, it is an ATP-dependent efflux pump with broad substrate specificity. It exists in animals, fungi and bacteria and likely evolved as a defense mechanism against harmful substances.

MDR1 is expressed in the intestinal epithelium where it pumps xenobiotics (such as toxins or drugs) back into the intestinal lumen, in liver cells where it pumps them into bile ducts, in the cells of the proximal tubule of the kidney where it pumps them into urine-conducting ducts, and in the capillary endothelial cells composing the blood-brain barrier and blood-testis barrier, where it pumps them back into the capillaries. Some cancer cells also express large amounts of MDR1, and this expression is thought to contribute to making these cancers multi-drug resistant.

ALDHA1 (Aldehyde dehydrogenase 1 isoform A1, also known as ALDH1A1 or retinaldehyde dehydrogenase 1 (RALDH1)) is an enzyme that in humans is encoded by the ALDH1A1 gene. ALDHA1 is one of 19 aldehyde dehydrogenase isoforms known to be expressed in human tissues and is a detoxifying enzyme responsible for oxidizing aldehydes to carboxylic acids. Expression of ALDHA1 in normal tissue is found mainly in the epithelium of testis, brain, eye, liver, and kidney. The enzyme also is expressed in high levels in hematopoietic and neural stem cells and plays a role in the differentiation of hematopoietic and neural stem cells via the oxidation of retinal to retinoic acid a key developmental regulator.

In some studies ALDHA1 expression was found to correlate with higher tumor grade but not with known indicators of cancer stage and metastasis. Other studies reported a correlation between ALDH1A1 expression and worsened outcomes in inflammatory breast cancer, while still other studies concluded that ALDHA3, but not ALDHA1, was predictive of metastasis in breast cancer. See: Marcato et al., Stem Cells; 29:32-45 (2011).

The most widely-used methodology presently applied to determine protein presence in cancer patient tissue, especially FFPE tissue, is immunohistochemistry (IHC). IHC methodology uses an antibody to detect the protein of interest. The results of an IHC test are most often interpreted by a pathologist or histotechnologist. This interpretation is subjective and does not provide quantitative data that are predictive of sensitivity to therapeutic agents that target specific oncoprotein targets. Thus, an IHC test cannot determine whether or not a cell population obtained from a liver mass is from a benign hepatocellular adenoma, a pre-malignant hepatocellular dysplastic nodule, or a malignant hepatocellular carcinoma.

Studies involving other IHC assays, such as the Her2 IHC test, suggest the results obtained from such tests may be wrong or misleading. This is likely because different laboratories use different rules for classifying positive and negative IHC status. Each pathologist running a test also may use different criteria to decide whether the results are positive or negative. In most cases, this happens when the test results are borderline, i.e. the results are neither strongly positive nor strongly negative. In other cases, tissue from one area of cancer tissue can test positive while tissue from a different area of the cancer tests negative.

Inaccurate IHC test results may mean that patients can be misdiagnosed with and thus do not receive the best possible care. If all or part of a cancer is positive for a specific target oncoprotein but test results classify it as negative, physicians are unlikely to implement the correct therapeutic treatment, even though the patient could potentially benefit from agents that target the oncoprotein. If a cancer is oncoprotein target negative but test results classify it as positive, physicians may use a specific therapeutic treatment, even though the patient is not only unlikely to receive any benefit but also is exposed to the agent's secondary risks.

Thus there is great clinical value in the ability to correctly evaluate quantitative levels of the MET, DHFR, MDR1, and ALDHA1 proteins in a liver mass, especially masses that are tumors, so that the patient will have the greatest chance of receiving a successful treatment regimen while reducing unnecessary toxicity and other side effects.

Detection of peptides and determining quantitative levels of specified MET, DHFR, MDR1, and ALDHA1 fragment peptides are determined in a mass spectrometer by the SRM/MRM methodology, in which the SRM/MRM signature chromatographic peak area of each peptide is determined within a complex peptide mixture present in a Liquid Tissue lysate (see U.S. Pat. No. 7,473,532, as described above). Quantitative levels of the analyzed proteins are then determined by the SRM/MRM methodology whereby the SRM/MRM signature chromatographic peak area of an individual specified peptide from each of the proteins in one biological sample is compared to the SRM/MRM signature chromatographic peak area of a known amount of a “spiked” internal standard for each of the individual specified fragment peptides.

In one embodiment, the internal standard is a synthetic version of the same fragment peptides where the synthetic peptides contain one or more amino acid residues labeled with one or more heavy isotopes, such as ²H, ¹⁸O, ¹⁷O, ¹⁵N, ¹³C, or combinations thereof. Such isotope labeled internal standards are synthesized so that mass spectrometry analysis generates a predictable and consistent SRM/MRM signature chromatographic peak that is different and distinct from the native fragment peptide chromatographic signature peaks and which can be used as comparator peaks. Thus when the internal standard is “spiked” in known amounts into a protein or peptide preparation from a biological sample and analyzed by mass spectrometry, the SRM/MRM signature chromatographic peak area of the native peptide is compared to the SRM/MRM signature chromatographic peak area of the internal standard peptide, and this numerical comparison indicates either the absolute molarity and/or absolute weight of the native peptide present in the original protein preparation from the biological sample. Quantitative data for fragment peptides are displayed according to the amount of protein analyzed per sample.

In order to develop the SRM/MRM assay for the fragment peptides additional information beyond simply the peptide sequence needs to be utilized by the mass spectrometer. That additional information is used to direct and instruct the mass spectrometer, (e.g., a triple quadrupole mass spectrometer) to perform the correct and focused analysis of the specified fragment peptides. An SRM/MRM assay may be effectively performed on a triple quadrupole mass spectrometer. That type of a mass spectrometer may be considered to be one of the most suitable instruments for analyzing a single isolated target peptide within a very complex protein lysate that may consist of hundreds of thousands to millions of individual peptides from all the proteins contained within a cell. The additional information provides the mass spectrometer, such as a triple quadrupole mass spectrometer, with the correct directives to allow analysis of a single isolated target peptide within a very complex protein lysate. SRM/MRM assays also can be developed and performed on other types of mass spectrometer, including MALDI, ion trap, ion trap/quadrupole hybrid, or triple quadrupole instruments, but presently the most advantageous instrument platform for SRM/MRM assay is often considered to be a triple quadrupole instrument platform. The additional information about target peptides in general, and in particular about the specified fragment peptides, may include one or more of the mono isotopic mass of each peptide, its precursor charge state, the precursor m/z value, the m/z transition ions, and the ion type of each transition ion.

Because both nucleic acids and protein can be analyzed from the same Liquid Tissue™ biomolecular preparation it is possible to generate additional information about disease diagnosis and drug treatment decisions from the nucleic acids in the same sample from which the measured proteins were analyzed. For example, if a measured protein, such as ALDHA1, is expressed by certain cells, such as liver cells, at increased levels when assayed by SRM, the data can provide information about the state of the cells and their potential for uncontrolled growth, potential drug resistance, and the development of cancers can be obtained. At the same time, information about the status of the corresponding genes and/or the nucleic acids and proteins they encode (e.g., mRNA molecules and their expression levels or splice variations) can be obtained from nucleic acids present in the same Liquid Tissue™ biomolecular preparation and can be assessed simultaneously with the SRM analysis of protein(s). Any gene and/or nucleic acid not from one of the measured proteins and which is present in the same biomolecular preparation can be assessed simultaneously to the SRM analysis of the measured protein(s). In one embodiment, information about one or more of ALDHA1, MDR1, MET and/or DHFR, and/or one, two, three, four or more additional proteins may be assessed by examining the nucleic acids encoding those proteins. Those nucleic acids can be examined, for example, by one or more, two or more, or three or more of: sequencing methods, polymerase chain reaction methods, restriction fragment polymorphism analysis, identification of deletions, insertions, and/or determinations of the presence of mutations, including but not limited to, single base pair polymorphisms, transitions, transversions, or combinations thereof.

Methods of Diagnosing a Hepatocellular Mass

To measure differential protein expression and to determine an appropriate reference level for protein quantitation, 22 liver mass samples were obtained from a cohort of patients suffering from liver disease: 8 benign hepatocellular adenomas, 5 pre-malignant hepatocellular dysplastic nodules, and 9 malignant hepatocellular carcinomas. The liver samples were formalin-fixed using standard methods and levels of 24 proteins were determined in the samples, including the MET, DHFR, MDR1, and ALDHA1 proteins using the methods as described above. The original diagnosis of these samples was performed by a trained pathologist using standard histological analysis via hematoxylin/eosin staining. A suitable reference level was determined using statistical methods that are well known in the art, for example by determining the lowest p value of a log rank test. Once a reference level was determined it was used to identify those patients whose protein expression levels indicate that a liver mass was a benign hepatocellular adenoma, a pre-malignant hepatocellular dysplastic nodule, or a malignant hepatocellular carcinoma.

Levels of MET, DHFR, MDR1, and ALDHA1 proteins in patient liver mass samples are typically expressed in amol/μg, although other units can be used. The skilled artisan will recognize that a reference level can be expressed as a range around a central value, for example, +/−250, 150, 100, 50 or 25 amol/μg. In the data presented in the figures and tables a suitable reference level for the MET protein was found to be 291 amol/μg. However, the skilled artisan will recognize that levels higher or lower than these reference levels can be selected based on clinical results and experience.

1. A method of diagnosing a hepatocellular mass comprising:
(a) quantifying the levels of one or more specified MET, DHFR, MDR1, and ALDHA1 fragment peptides in a protein digest prepared from a tissue sample obtained from the liver of a patient and calculating the level of said one or more MET, DHFR, MDR1, and ALDHA1 peptides in said sample by selected reaction monitoring using mass spectrometry;
(b) comparing the level of said one or more MET, DHFR, MDR1, and ALDHA1 fragment peptides to a pair of reference levels, wherein
a MET fragment peptide is compared to a MET benign hepatocellular adenoma reference level and a MET pre-malignant dysplastic nodule or malignant carcinoma reference level,
a DHFR fragment peptide is compared to a DHFR benign hepatocellular adenoma reference level and a DHFR pre-malignant dysplastic nodule or malignant carcinoma reference level,
an MDR1 fragment peptide is compared to an MDR1 benign hepatocellular adenoma reference level and an MDR1 pre-malignant dysplastic nodule or malignant carcinoma reference level, and
an ALDHA1 fragment peptide is compared to an ALDHA1 benign hepatocellular adenoma reference level and an ALDHA1 pre-malignant dysplastic nodule or malignant carcinoma reference level;
(c) determining that the liver mass is a benign hepatocellular adenoma or a pre-malignant dysplastic nodule or malignant carcinoma, using the criteria wherein:
a level of MDR1 fragment peptide lower than said MDR1 benign hepatocellular adenoma reference level indicates that the mass is a benign hepatocellular adenoma;
a level of ALDHA1 fragment peptide lower than said ALDHA1 benign hepatocellular adenoma reference level indicates that the mass is a benign hepatocellular adenoma;
a level of MDR1 fragment peptide higher than said MDR1 ALDHA1 pre-malignant dysplastic nodule or malignant carcinoma reference level indicates that the mass is a pre-malignant dysplastic nodule or malignant carcinoma reference;
a level of ALDHA1 fragment peptide higher than said ALDHA1 pre-malignant dysplastic nodule or malignant carcinoma reference level indicates that the mass is a pre-malignant dysplastic nodule or malignant carcinoma reference;
a level of MET fragment peptide higher than said MET benign hepatocellular adenoma reference level indicates that the mass is a benign hepatocellular adenoma;
a level of DHFR fragment peptide higher than said DHFR benign hepatocellular adenoma reference level indicates that the mass is a benign hepatocellular adenoma;
a level of MET fragment peptide lower than said MET pre-malignant dysplastic nodule or malignant carcinoma reference level indicates that the mass is a pre-malignant dysplastic nodule or malignant carcinoma reference; and
a level of DHFR fragment peptide lower than said pre-malignant dysplastic nodule or malignant carcinoma reference level indicates that the mass is a pre-malignant dysplastic nodule or malignant carcinoma.

2. The method of claim 1 wherein said MET benign hepatocellular adenoma reference level is 291 amol/μg., +/−290 amol/μg, of biological sample protein analyzed.

3. The method of claim 1 wherein said DHFR benign hepatocellular adenoma reference level is 301 amol/μg., +/−300 amol/μg, of biological sample protein analyzed,

4. The method of claim 1 wherein said MDR1 benign hepatocellular adenoma reference is 81 amol/μg., +/−80 amol/μg, of biological sample protein analyzed.

5. The method of claim 1 wherein said ALDHA1 benign hepatocellular adenoma reference level is 20,586 amol/μg., +/−20,585 amol/μg, of biological sample protein analyzed.

6. The method of claim 1 wherein said MET pre-malignant dysplastic nodule or malignant hepatocellular carcinoma reference level is 187 amol/μg., +/−186 amol/μg, of biological sample protein analyzed.

7. The method of claim 1 wherein said DHFR pre-malignant dysplastic nodule or malignant hepatocellular carcinoma reference level is 162 amol/μg., +/−161 amol/μg, of biological sample protein analyzed.

8. The method of claim 1 wherein said MDR1 pre-malignant dysplastic nodule or malignant hepatocellular carcinoma reference level is 304 amol/μg., +/−303 amol/μg, of biological sample protein analyzed.

9. The method of claim 1 wherein said ALDHA1 pre-malignant dysplastic nodule or malignant hepatocellular carcinoma reference level is 32,500 amol/μg., +/−32,499 amol/μg, of biological sample protein analyzed.

10. The method of claim 9 wherein said protein digest comprises a protease digest.

11. The method of claim 10, wherein said protein digest comprises a trypsin digest.

12. The method of claim 1, wherein mass spectrometry comprises tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry.

13. The method of claim 12, wherein the mode of mass spectrometry used is Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

14. The method of claim 1, wherein the tumor sample is a cell, collection of cells, or a solid tissue.

15. The method of claim 14, wherein the tumor sample is formalin fixed solid tissue.

16. The method of claim 15, wherein the tissue is paraffin embedded tissue.

17. The method of claim 1, wherein quantifying the specified MET, DHFR, MDR1, and ALDHA1 fragment peptides comprises determining the amount of the specified MET, DHFR, MDR1, and ALDHA1 fragment peptides in said sample by comparing to a spiked internal standard peptide of known amount, wherein both the native peptides in the biological sample and the internal standard peptides correspond to the same amino acid sequence of the specified MET, DHFR, MDR1, and ALDHA1 fragment peptides.

18. The method of claim 17 wherein the internal standard peptide is an isotopically labeled peptide.

19. The method of claim 18 wherein the isotopically labeled internal standard peptide comprises one or more heavy stable isotopes selected from ¹⁸O, ¹⁷O, ¹⁵N, ¹³C, ²H or combinations thereof.

20. The method of claim 17, wherein detecting and quantitating the specified MET, DHFR, MDR1, and ALDHA1 fragment peptides is combined with detecting and quantitating other peptides from other proteins in multiplex so that the diagnostic decision about a hepatocellular mass is based upon specific levels of the specified MET, DHFR, MDR1, and ALDHA1 fragment peptides in combination with other peptides/proteins in the biological sample.

21. The method of claim 1 wherein when said level of said specified MDR1 and/or ALDHA1 peptides is higher than said reference level, then said therapeutic strategy may comprise treating the patient with chemotherapy.

22. The method of claim 1 wherein when said level of said specified MET and/or DHFR peptides is lower than said reference level, then said therapeutic strategy may comprise treating the patient with chemotherapy.

23. The method of claim 1 wherein when said level of said specified MET and/or DHFR peptides is higher than said reference level, then said therapeutic strategy comprises treating the patient with surgical resection of said liver mass.

24. The method of claim 1 wherein when said level of said specified MDR1 and/or ALDHA1 peptides is lower than said reference level, then said therapeutic strategy comprises treating the patient with surgical resection of said liver mass.

25. The method of claim 1 wherein measuring the level of the MET protein in a human biological sample of formalin-fixed tissue, comprises detecting and/or quantifying the amount of a specified fragment peptide in a protein digest prepared from said human biological sample using mass spectrometry; and calculating the level of MET protein in said sample; wherein the MET fragment peptide is SEQ ID NO:1, and wherein said level is a relative level or an absolute level.

26. The method of claim 1 wherein measuring the level of the DHFR protein in a human biological sample of formalin-fixed tissue, comprises detecting and/or quantifying the amount of a specified fragment peptide in a protein digest prepared from said human biological sample using mass spectrometry; and calculating the level of DHFR protein in said sample; wherein the DHFR fragment peptide is SEQ ID NO:2, and wherein said level is a relative level or an absolute level.

27. The method of claim 1 wherein measuring the level of the MDR1 protein in a human biological sample of formalin-fixed tissue, comprises detecting and/or quantifying the amount of a specified fragment peptide in a protein digest prepared from said human biological sample using mass spectrometry; and calculating the level of MDR1 protein in said sample; wherein the MDR fragment peptide is SEQ ID NO:3 and/or SEQ ID NO:4, and wherein said level is a relative level or an absolute level.

28. The method of claim 1 wherein measuring the level of the ALDHA1 protein in a human biological sample of formalin-fixed tissue, comprising detecting and/or quantifying the amount of a specified fragment peptide in a protein digest prepared from said human biological sample using mass spectrometry; and calculating the level of ALDH1 protein in said sample; wherein the ALDH1 fragment peptide is SEQ ID NO:5 and/or SEQ ID NO:6 and wherein said level is a relative level or an absolute level.

29. The method of claim 1, wherein quantifying said fragment peptide comprises comparing the amount of said fragment peptide in one biological sample to the amount of the same fragment peptide in a different and separate biological sample.

30. A method for measuring the level of the human Aldehyde Dehydrogenase 1 isoform A1 (ALDHA1) protein in a human biological sample of formalin-fixed tissue, comprising detecting and quantifying the amount of at least one ALDHA1 fragment peptide in a protein digest prepared from said human biological sample using mass spectrometry; and calculating the level of ALDHA1 protein in said sample; wherein said at least one ALDHA1 fragment peptide is the peptide of SEQ ID NO:5 or SEQ ID NO:6, and wherein said level is a relative level or an absolute level.

31. The method according to claim 30 wherein said peptide is the peptide of SEQ ID NO:5.

32. The method according to claim 30 wherein said peptide is the peptide of SEQ ID NO:6.

33. The method according to claim 30 wherein the peptides of SEQ ID NO:5 and SEQ ID NO:6 are detected.

34. The method of claim 30, further comprising the step of fractionating said protein digest prior to detecting and/or quantifying the amount of said at least one ALDHA1 fragment peptide.

35. The method of claim 30, wherein said protein digest comprises a protease digest.

36. The method of claim 30, wherein detecting and quantifying the amount of said at least one ALDHA1 fragment peptide in the protein digest indicates the presence of ALDHA1 protein and an association with cancer in the subject.

37. The method of claim 36, further comprising correlating the results of said detecting and quantifying the amount of said at least one ALDHA1 fragment peptide, or the level of said ALDHA1 protein to the diagnostic stage/grade/status of the cancer.

38. The method of claim 37, wherein correlating the results of said detecting and quantifying the amount of said at least one ALDHA1 fragment peptide, or the level of said ALDHA1 protein to the diagnostic stage/grade/status of the cancer is combined with detecting and/or quantifying the amount of other proteins or peptides from other proteins in a multiplex format to provide additional information about the diagnostic stage/grade/status of the cancer.

39. The method of claim 30, further comprising administering to the patient from which said biological sample was obtained a therapeutically effective amount of a therapeutic agent, wherein the therapeutic agent and/or amount of the therapeutic agent administered is based upon the amount of said ALDHA1 fragment peptide or the level of ALDHA1 protein.

40. The method of claim 30, wherein said therapeutic agent binds the ALDHA1 protein and/or inhibits its biological activity.

41. A method for measuring the level of the human multidrug resistance protein 1 (MDR1) protein in a human biological sample of formalin-fixed tissue, comprising detecting and quantifying the amount of at least one MDR1 fragment peptide in a protein digest prepared from said human biological sample using mass spectrometry; and calculating the level of MDR1 protein in said sample; wherein said at least one MDR1 fragment peptide is the peptide of SEQ ID NO:3 or SEQ ID NO:4, and wherein said level is a relative level or an absolute level.

42. The method according to claim 41 wherein said peptide is the peptide of SEQ ID NO:4.

43. The method according to claim 41 wherein said peptide is the peptide of SEQ ID NO:4.

44. The method according to claim 41 wherein the peptides of SEQ ID NO:3 and SEQ ID NO:4 are detected.

45. The method of claim 41, further comprising the step of fractionating said protein digest prior to detecting and quantifying the amount of said at least one MDR1 fragment peptide.

46. The method of claim 41, wherein said protein digest comprises a protease digest.

47. The method of claim 41, wherein detecting and quantifying the amount of said at least one MDR1 fragment peptide in the protein digest indicates the presence of MDR1 protein and an association with cancer in the subject.

48. The method of claim 47, further comprising correlating the results of said detecting and quantifying the amount of said at least one MDR1 fragment peptide, or the level of said MDR1 protein to the diagnostic stage/grade/status of the cancer.

49. The method of claim 48, wherein correlating the results of said detecting and quantifying the amount of said at least one MDR1 fragment peptide, or the level of said MDR1 protein to the diagnostic stage/grade/status of the cancer is combined with detecting and/or quantifying the amount of other proteins or peptides from other proteins in a multiplex format to provide additional information about the diagnostic stage/grade/status of the cancer.

50. The method of claim 41, further comprising administering to the patient from which said biological sample was obtained a therapeutically effective amount of a therapeutic agent, wherein the therapeutic agent and/or amount of the therapeutic agent administered is based upon the amount of said MDR1 fragment peptide or the level of MDR1 protein.

51. The method of claim 51, wherein said therapeutic agent binds the MDR1 protein and/or inhibits its biological activity
